(19) United States
(12) Patent Application Publication
(10) Pub. No.: US 2025/0051509 A1
(43) Pub. Date: Feb. 13, 2025

(54) CURABLE RESIN COMPOSITION, VARNISH, CURED PRODUCT, AND METHOD FOR PRODUCING CURED PRODUCT

(71) Applicant: HONSHU CHEMICAL INDUSTRY CO., LTD., Chuo-ku, Tokyo (JP)

(72) Inventor: Yoshimi UTAKA, Wakayama-shi (JP)

(21) Appl. No.: 18/685,462

(22) PCT Filed: Aug. 9, 2022
(86) PCT No.: PCT/JP2022/030384

§ 371 Date: Feb. 21, 2024

(30) Foreign Application Priority Data
Sep. 10, 2021 (JP) 2021-147514

(51) Int. Cl. C08G 59/24; C08G 59/40; C08G 59/68; C08J 3/24; C08K 5/378; C09D 7/20; C09D 7/63; C09D 163/00

(52) U.S. Cl. CPC C08G 59/245; C08G 59/4042; C08G 59/686; C08G 59/687; C08G 59/688; C08J 3/247; C08K 5/378; C09D 7/20; C09D 7/63; C09D 163/00; C08G 2150/20; C08J 2363/02

(57) ABSTRACT

A curable resin composition contains 100 parts by weight of a component (A) and 5 to 2000 parts by weight of at least one of a component (B) and a component (C): (A) a benzoxazine compound having a thiol group; (B) a compound having a three- or four-membered ring cyclic ether group; (C) a compound having a reactive group including a carbon-carbon double bond or a carbon-carbon triple bond. The curable composition is intended to suppress an odorous volatile component (sulfur-containing volatile component) generated during the production of a cured product using the curable resin composition.

### TECHNICAL FIELD

The present invention relates to a curable resin composition containing a specific curing agent and a benzoxazine compound having benzoxazine rings at both ends of a linking group and further having a thiol group, a varnish, a cured product, and a method for producing a cured product.

### BACKGROUND ART

Benzoxazine compounds are known as thermosetting resin raw materials that, when heated, undergo ring-opening polymerization of a benzoxazine ring to cure without producing any volatile by-products, and are used as raw materials of a molded body usable as a material for an insulating substrate, a liquid crystal alignment agent, a resin composition for semiconductor sealing, and the like.

On the other hand, benzoxazine compounds typically have relatively high curing temperatures, and to achieve lower polymerization temperatures, catalysts, polymerization accelerators, and highly reactive benzoxazine compounds have recently been developed. Among the highly reactive benzoxazine compounds, a hydroxy-functionalized benzoxazine composition having a structure in which a hydroxy group is introduced has been reported (PTL 1).

However, to lower the temperature during the process of molding a thermosetting resin, thereby achieving higher efficiency due to reduced time of heating and cooling and saved energy and suppressing material thermal degradation due to exposure to high temperature during polymerization, excellent materials that can cure under low-temperature conditions are demanded.

Under these circumstances, the present inventors have invented, as a novel benzoxazine compound that can cure under low-temperature conditions, a benzoxazine compound having benzoxazine rings at both ends of a linking group and further having a thiol group and filed a patent application (PTL 2).

### CITATION LIST

### Patent Literature

PTL 1: Japanese Unexamined Patent Application Publication No. 2011-530570

PTL 2: Japanese Patent Application No. 2021-013397

### SUMMARY OF INVENTION

### Technical Problem

As a result of studies on the production of a cured product using a benzoxazine compound represented by general formula (1) below among the above benzoxazine compounds having a thiol group, when a curable resin composition is cured under the curing conditions described in PTL 2 above, a weight loss after curing and generation of an odorous volatile component have sometimes occurred.

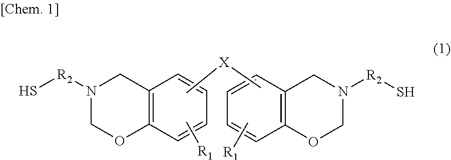

(In the formula, each R₁ independently represents a hydrogen atom or an alkyl group having 1 to 6 carbon atoms, each R₂ independently represents a linear, branched, or aliphatic-ring-containing alkylene group having 1 to 10 carbon atoms, and X represents a single bond, an oxygen atom, a sulfur atom, a sulfonyl group, a carbonyl group, or a divalent group represented by formula 1a or formula 1b below.)

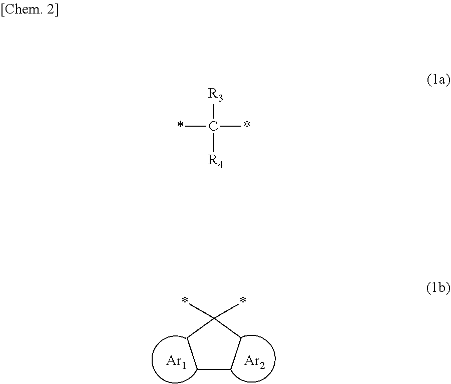

(In formula 1a and formula 1b, R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)

Generation of a volatile component during the production of a cured product will result in formation of vacuum cavities (voids) inside the cured product, which is disadvantageous, for example, in that these areas become fragile to decrease mechanical strength, thus failing to provide a good cured product and that the cured product shrinks upon volatilization and has poor dimensional stability. In addition, the odorous volatile component is undesirable in terms of, for example, safety, health, and environment and necessitates measures such as introduction of equipment that does not allow leakage out of the system.

The present inventors have discovered that the odorous volatile component is generated as a result of decomposition of the benzoxazine compound represented by general formula (1) during heat curing and is a sulfur-containing volatile component. It is presumed that the generation of the odorous volatile component (sulfur-containing volatile component) is due to a change of the thiol group moiety of the benzoxazine structure as represented by the following formula.

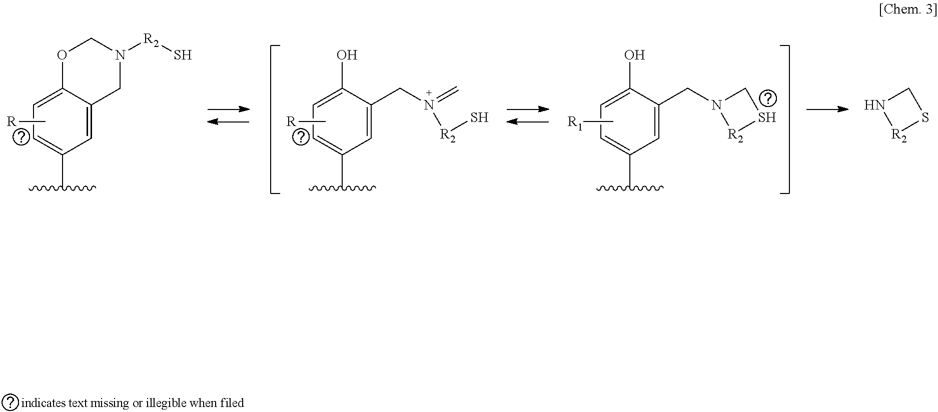

(In the formula, R₁ and R₂ are as defined in general formula 1.)

For example, when R₂ of the benzoxazine compound represented by general formula (1) is an ethylene group, the odorous volatile component (sulfur-containing volatile component) generated during curing is thiazolidine.

Against the above background, an object of the present invention is to provide means for suppressing an odorous volatile component (sulfur-containing volatile component) generated during the production of a cured product using a curable resin composition containing the benzoxazine compound represented by general formula (1).

In addition, since the glass transition temperature of a cured product obtained using only the benzoxazine compound having a thiol group described in PTL 2 above is lower than those of existing F-a type benzoxazines, another object of the present invention is to provide a cured product having further improved heat resistance.

### Solution to Problem

To achieve the above objects, the present inventors have conducted intensive studies and found that a composition containing a benzoxazine compound having a thiol group and a compound having a specific reactive group can suppress the generation of an odorous volatile component (sulfur-containing volatile component) during curing, thereby completing the present invention.

The inventors have further found that a cured product obtained therefrom has significantly improved heat resistance as compared with a cured product obtained using only the benzoxazine compound having a thiol group.

The present invention is as follows.

1. A curable resin composition containing 100 parts by weight of a component (A) below and 5 to 2000 parts by weight of at least one of a component (B) and a component (C) below.
(A): A benzoxazine compound represented by general formula (1) below.

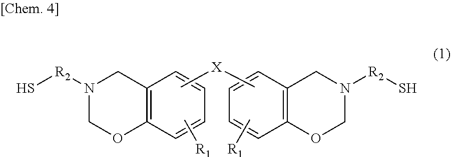

(In the formula, each R₁ independently represents a hydrogen atom or an alkyl group having 1 to 6 carbon atoms, each R₂ independently represents a linear, branched, or aliphatic-ring-containing alkylene group having 1 to 10 carbon atoms, and X represents a single bond, an oxygen atom, a sulfur atom, a sulfonyl group, a carbonyl group, or a divalent group represented by formula 1a or formula 1b below.)

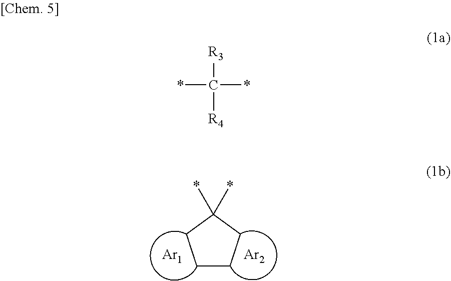

(In formula 1a and formula 1b, R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)
(B): A compound having a three- or four-membered ring cyclic ether group.
(C): A compound having a reactive group including a carbon-carbon double bond or a carbon-carbon triple bond.
   2. The curable resin composition according to 1., containing a component (D) below.
(D): A curing reaction catalyst
   3. The curable resin composition according to 2., wherein the curing reaction catalyst is an acid catalyst.
   4. The curable resin composition according to any one of 1. to 3., further containing a component (E) below.
(E): A filler
   5. A varnish containing the curable resin composition according to 1, and a component (F) below.
(F): An organic solvent
   6. A cured product obtained by curing the curable resin composition according to 1.
   7. A method for producing a cured product containing a component (A) below, the method including curing a curable resin composition containing the component (A) and at least one of a component (B) and a component (C) below.
(A): A benzoxazine compound represented by general formula (1) below.

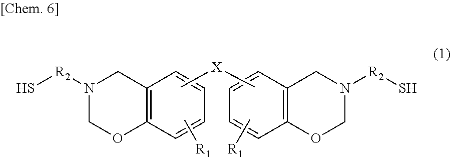

(In the formula, each R₁ independently represents a hydrogen atom or an alkyl group having 1 to 6 carbon atoms, each R₂ independently represents a linear, branched, or aliphatic-ring-containing alkylene group having 1 to 10 carbon atoms, and X represents a single bond, an oxygen atom, a sulfur atom, a sulfonyl group, a carbonyl group, or a divalent group represented by formula 1a or formula 1b below.)

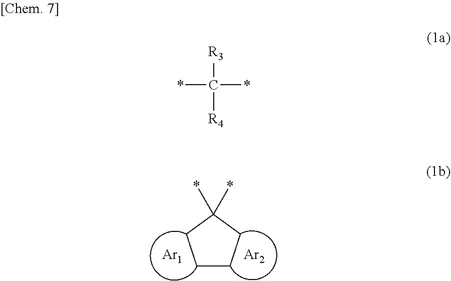

(In formula 1a and formula 1b, R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)
(B): A compound having a three- or four-membered ring cyclic ether group.
(C): A compound having a reactive group including a carbon-carbon double bond or a carbon-carbon triple bond.
   8. The method for producing a cured product according to 7., wherein the method for producing a cured product includes a pre-curing step under a temperature condition in a range of 60° C. to 150° C. and a curing step under a temperature condition in a range of 150° C. to 240° C.
   9. The method for producing a cured product according to 7, or 8., wherein the curable resin composition further contains a component (D) below.
(D): A curing reaction catalyst
   10. The method for producing a cured product according to 9., wherein the curing reaction catalyst is an acid catalyst.

### Advantageous Effects of Invention

The curable resin composition containing a benzoxazine compound having a thiol group and a compound having a specific reactive group according to the present invention can suppress the generation of an odorous volatile component (sulfur-containing volatile component) during curing.

Furthermore, the cured product obtained from the curable resin composition has high heat resistance.

In the method for producing a cured product using the curable resin composition containing a benzoxazine compound having a thiol group according to the present invention, the curable resin composition containing a benzoxazine compound having a thiol group and a compound having a specific reactive group is cured, whereby the generation of an odorous volatile component (sulfur-containing volatile component) during the production can be suppressed.

### BRIEF DESCRIPTION OF DRAWINGS

FIG. 1 shows a chart of dynamic viscoelasticity analysis (DMA) of a cured product obtained in Example 14.

FIG. 2 shows a chart of dynamic viscoelasticity analysis (DMA) of a cured product obtained in Comparative Example 3.

### DESCRIPTION OF EMBODIMENTS

### Curable Resin Composition According to Present Invention

A curable resin composition according to the present invention contains 100 parts by weight of a component (A) and 5 to 2000 parts by weight of at least one of a component (B) and a component (C) below, the components being described later.

### <Component (A): Benzoxazine Compound Represented by General Formula (1)>

The component (A) is a benzoxazine compound represented by general formula (1) below.

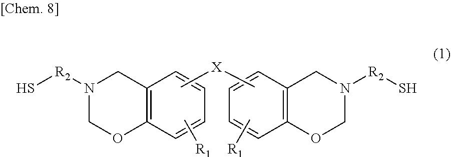

(In the formula, each R₁ independently represents a hydrogen atom or an alkyl group having 1 to 6 carbon atoms, each R₂ independently represents a linear, branched, or aliphatic-ring-containing alkylene group having 1 to 10 carbon atoms, and X represents a single bond, an oxygen atom, a sulfur atom, a sulfonyl group, a carbonyl group, or a divalent group represented by formula 1a or formula 1b below.)

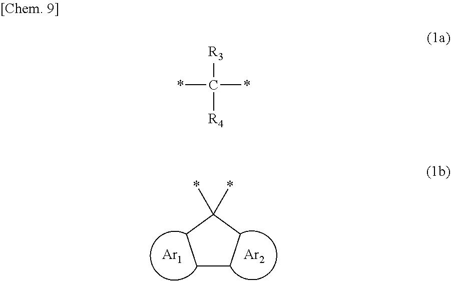

(In formula 1a and formula 1b, R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)

R₁ in general formula (1) at each occurrence is preferably a hydrogen atom or an alkyl group having 1 to 4 carbon atoms, more preferably a hydrogen atom or an alkyl group having 1 carbon atom (methyl group), particularly preferably a hydrogen atom. When R₁ is not a hydrogen atom, the bonding position thereof is preferably the ortho position on the benzene ring relative to the oxygen atom of each benzoxazine ring.

R₂ in general formula (1) at each occurrence is a linear, branched, or aliphatic-ring-containing alkylene group having 1 to 10 carbon atoms; specific examples include a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group. Of these, an ethylene group, a propane-1,2-diyl group, a propane-1,3-diyl group, and a butane-1,4-diyl group are particularly preferred.

Of these, R₂ is preferably a linear or branched alkylene group having 1 to 10 carbon atoms, more preferably a linear or branched alkylene group having 1 to 6 carbon atoms, still more preferably a linear or branched alkylene group having 1 to 4 carbon atoms, particularly preferably a linear or branched alkylene group having 2 to 4 carbon atoms.

When X in general formula (1) above is represented by formula (1a), R₃ and R₄, independently of each other, are more preferably hydrogen, an alkyl group having 1 to 6 carbon atoms, an alkyl halide group having 1 to 6 carbon atoms, or an aryl group having 6 to 12 carbon atoms, still more preferably hydrogen, an alkyl group having 1 to 4 carbon atoms, a trifluoromethyl group, or an aryl group having 6 to 8 carbon atoms, particularly preferably hydrogen, an alkyl group having 1 to 4 carbon atoms, or a phenyl group.

R₃ and R₄ may be bonded to each other to together form a cycloalkylidene group having 5 to 20 carbon atoms. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms.

Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), a cycloheptylidene group (7 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), and more preferred are a cyclohexylidene group (6 carbon atoms), a 3,3,5-trimethylcyclohexylidene group (9 carbon atoms), and a cyclododecanylidene group (12 carbon atoms).

When X in general formula (1) above is represented by formula (1b), Ar₁ and Ar₂ are preferably each independently a benzene ring or a naphthalene ring, and Ar₁ and Ar₂ are more preferably both benzene rings. For example, when Ar₁ and Ar₂ are both benzene rings, the group represented by formula (1b) is a fluorenylidene group.

The position of bonding of X in general formula (1) to the two benzoxazine rings is preferably the ortho or para position on the benzene ring relative to the oxygen atom of each benzoxazine ring.

As specific examples of the benzoxazine compound represented by general formula (1) according to the present invention, compounds (p-1) to (p-63) having the following chemical structures are shown. Of these, compounds (p-1) to (p-42), compounds (p-46) to (p-48), and compounds (p-52) to (p-63) are preferred, compounds (p-1) to (p-15), compounds (p-22) to (p-30), compounds (p-34) to (p-42), and compounds (p-52) to (p-63) are more preferred.

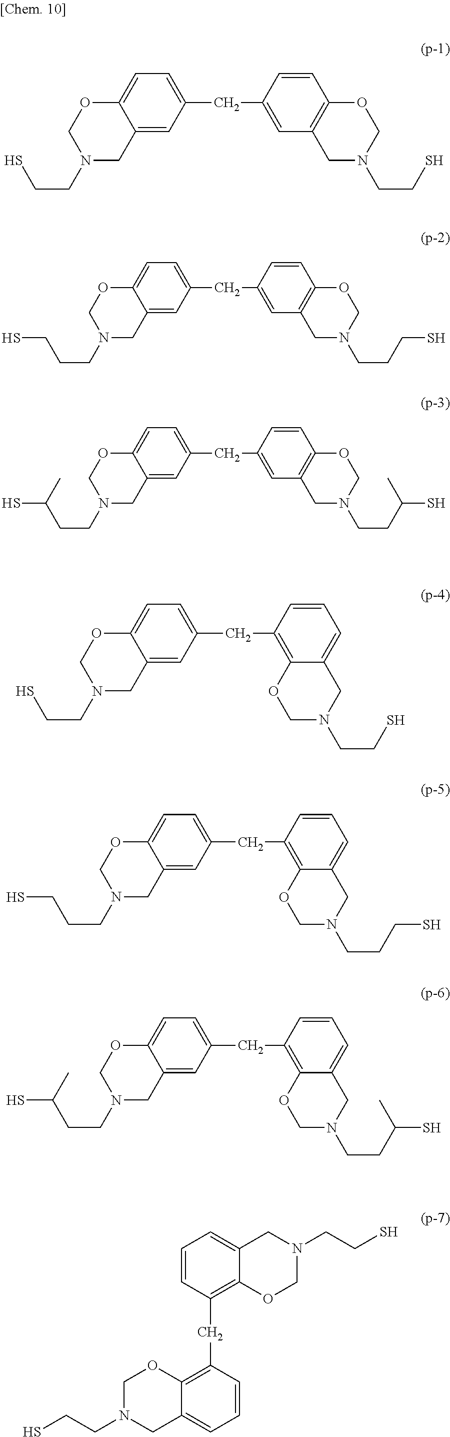

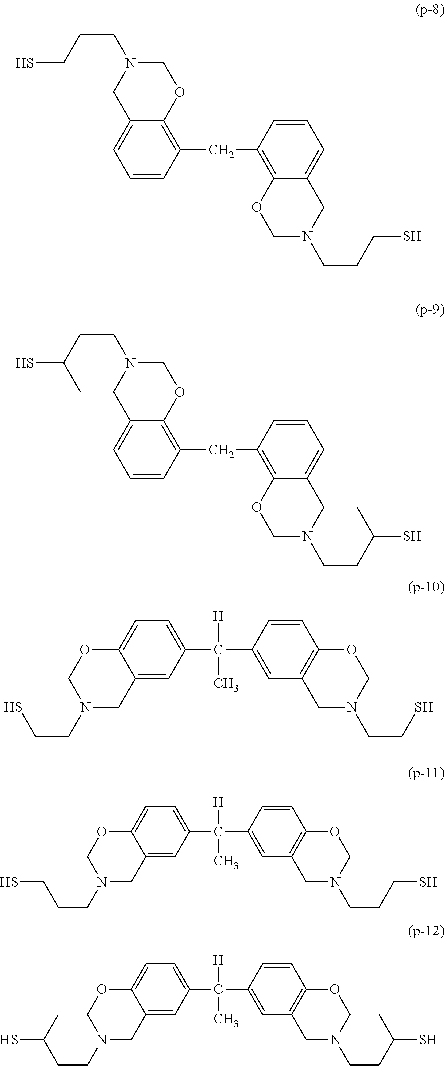 [SMILES: SCCCN1COc2c(Cc3cccc4c3OCN(CCCS)C4)cccc2C1 ; CC(S)CCN1COc2c(Cc3cccc4c3OCN(CCC(C)S)C4)cccc2C1 ; CC(c1ccc2c(c1)CN(CCS)CO2)c1ccc2c(c1)CN(CCS)CO2 ; CC(c1ccc2c(c1)CN(CCCS)CO2)c1ccc2c(c1)CN(CCCS)CO2 ; CC(S)CCN1COc2ccc(C(C)c3ccc4c(c3)CN(CCC(C)S)CO4)cc2C1]

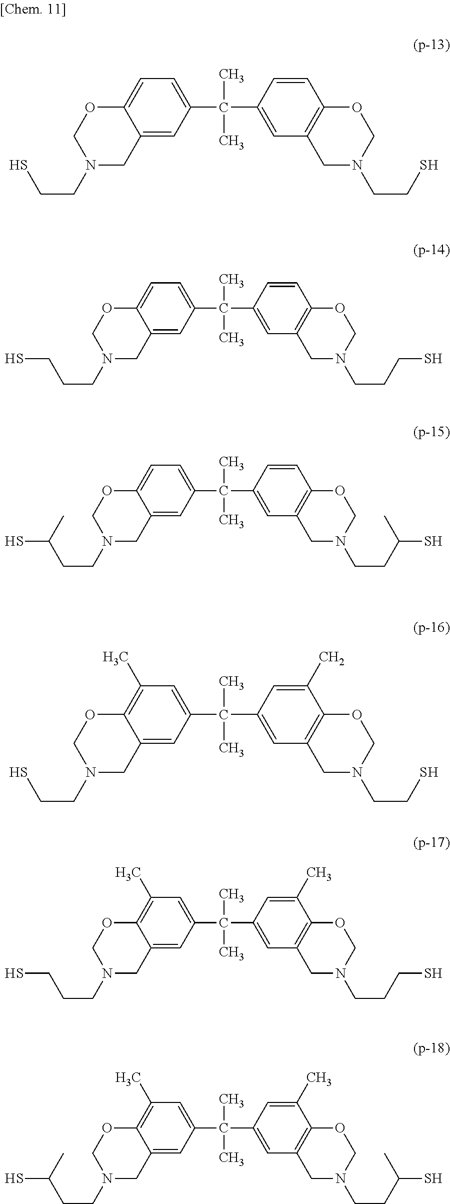 [SMILES: CC(C)(c1ccc2c(c1)CN(CCS)CO2)c1ccc2c(c1)CN(CCS)CO2 ; CC(C)(c1ccc2c(c1)CN(CCCS)CO2)c1ccc2c(c1)CN(CCCS)CO2 ; CC(S)CCN1COc2ccc(C(C)(C)c3ccc4c(c3)CN(CCC(C)S)CO4)cc2C1 ; Cc1cc(C(C)(C)c2cc(C)c3c(c2)CN(CCS)CO3)cc2c1OCN(CCS)C2 ; Cc1cc(C(C)(C)c2cc(C)c3c(c2)CN(CCCS)CO3)cc2c1OCN(CCCS)C2 ; Cc1cc(C(C)(C)c2cc(C)c3c(c2)CN(CCC(C)S)CO3)cc2c1OCN(CCC(C)S)C2]

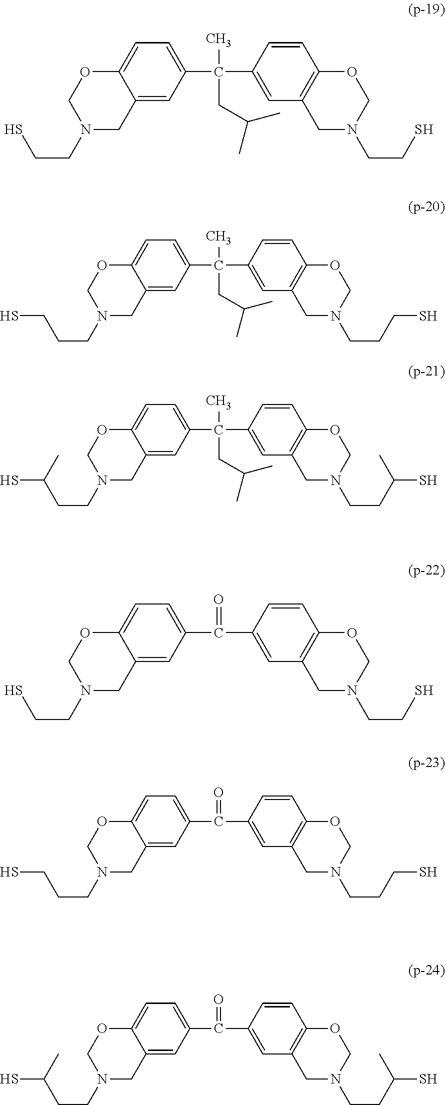 [SMILES: CC(C)CC(C)(c1ccc2c(c1)CN(CCS)CO2)c1ccc2c(c1)CN(CCS)CO2 ; CC(C)CC(C)(c1ccc2c(c1)CN(CCCS)CO2)c1ccc2c(c1)CN(CCCS)CO2 ; CC(C)CC(C)(c1ccc2c(c1)CN(CCC(C)S)CO2)c1ccc2c(c1)CN(CCC(C)S)CO2 ; O=C(c1ccc2c(c1)CN(CCS)CO2)c1ccc2c(c1)CN(CCS)CO2 ; O=C(c1ccc2c(c1)CN(CCCS)CO2)c1ccc2c(c1)CN(CCCS)CO2 ; CC(S)CCN1COc2ccc(C(=O)c3ccc4c(c3)CN(CCC(C)S)CO4)cc2C1]

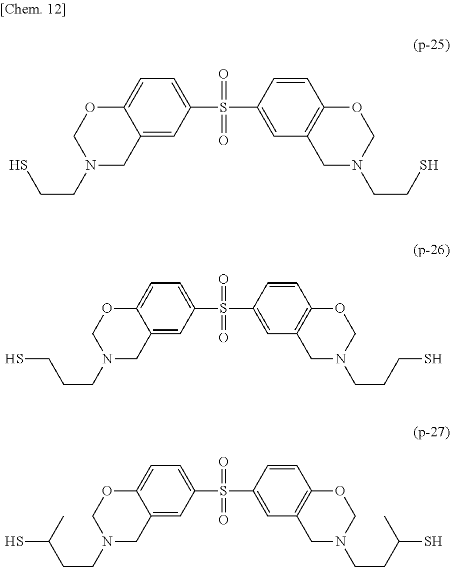 [SMILES: O=S(=O)(c1ccc2c(c1)CN(CCS)CO2)c1ccc2c(c1)CN(CCS)CO2 ; O=S(=O)(c1ccc2c(c1)CN(CCCS)CO2)c1ccc2c(c1)CN(CCCS)CO2 ; CC(S)CCN1COc2ccc(S(=O)(=O)c3ccc4c(c3)CN(CCC(C)S)CO4)cc2C1]

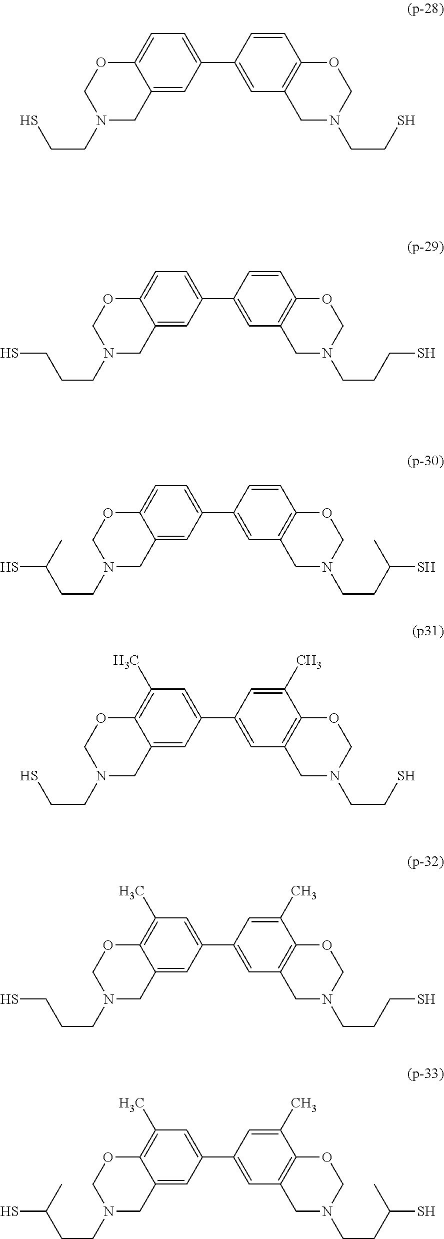 [SMILES: SCCN1COc2ccc(-c3ccc4c(c3)CN(CCS)CO4)cc2C1 ; SCCCN1COc2ccc(-c3ccc4c(c3)CN(CCCS)CO4)cc2C1 ; CC(S)CCN1COc2ccc(-c3ccc4c(c3)CN(CCC(C)S)CO4)cc2C1 ; Cc1cc(-c2cc(C)c3c(c2)CN(CCS)CO3)cc2c1OCN(CCS)C2 ; Cc1cc(-c2cc(C)c3c(c2)CN(CCCS)CO3)cc2c1OCN(CCCS)C2 ; Cc1cc(-c2cc(C)c3c(c2)CN(CCC(C)S)CO3)cc2c1OCN(CCC(C)S)C2]

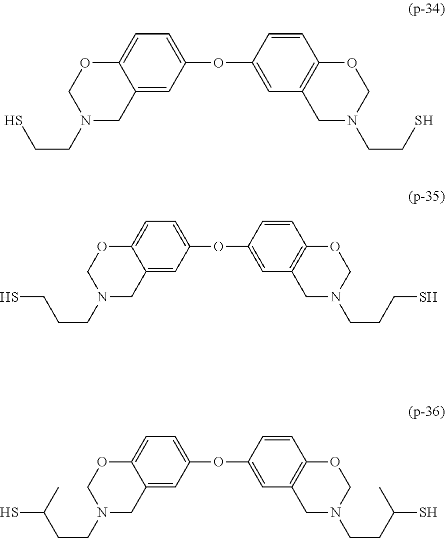 [SMILES: SCCN1COc2ccc(Oc3ccc4c(c3)CN(CCS)CO4)cc2C1 ; SCCCN1COc2ccc(Oc3ccc4c(c3)CN(CCCS)CO4)cc2C1 ; CC(S)CCN1COc2ccc(Oc3ccc4c(c3)CN(CCC(C)S)CO4)cc2C1]

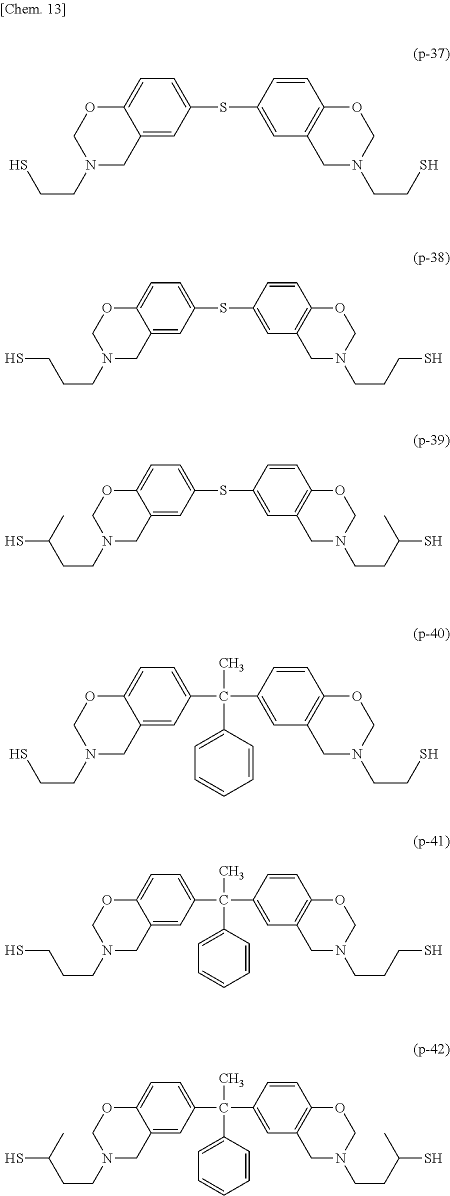 [SMILES: SCCN1COc2ccc(Sc3ccc4c(c3)CN(CCS)CO4)cc2C1 ; SCCCN1COc2ccc(Sc3ccc4c(c3)CN(CCCS)CO4)cc2C1 ; CC(S)CCN1COc2ccc(Sc3ccc4c(c3)CN(CCC(C)S)CO4)cc2C1 ; CC(c1ccccc1)(c1ccc2c(c1)CN(CCS)CO2)c1ccc2c(c1)CN(CCS)CO2 ; CC(c1ccccc1)(c1ccc2c(c1)CN(CCCS)CO2)c1ccc2c(c1)CN(CCCS)CO2 ; CC(S)CCN1COc2ccc(C(C)(c3ccccc3)c3ccc4c(c3)CN(CCC(C)S)CO4)cc2C1]

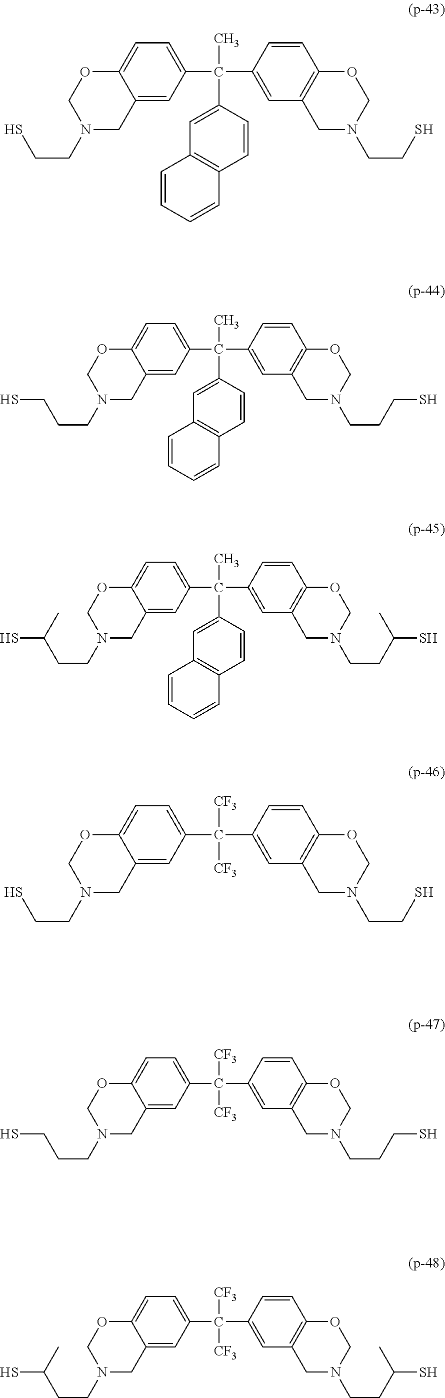 [SMILES: CC(c1ccc2c(c1)CN(CCS)CO2)(c1ccc2c(c1)CN(CCS)CO2)c1ccc2ccccc2c1 ; CC(c1ccc2c(c1)CN(CCCS)CO2)(c1ccc2c(c1)CN(CCCS)CO2)c1ccc2ccccc2c1 ; CC(S)CCN1COc2ccc(C(C)(c3ccc4c(c3)CN(CCC(C)S)CO4)c3ccc4ccccc4c3)cc2C1 ; FC(F)(F)C(c1ccc2c(c1)CN(CCS)CO2)(c1ccc2c(c1)CN(CCS)CO2)C(F)(F)F ; FC(F)(F)C(c1ccc2c(c1)CN(CCCS)CO2)(c1ccc2c(c1)CN(CCCS)CO2)C(F)(F)F ; CC(S)CCN1COc2ccc(C(c3ccc4c(c3)CN(CCC(C)S)CO4)(C(F)(F)F)C(F)(F)F)cc2C1]

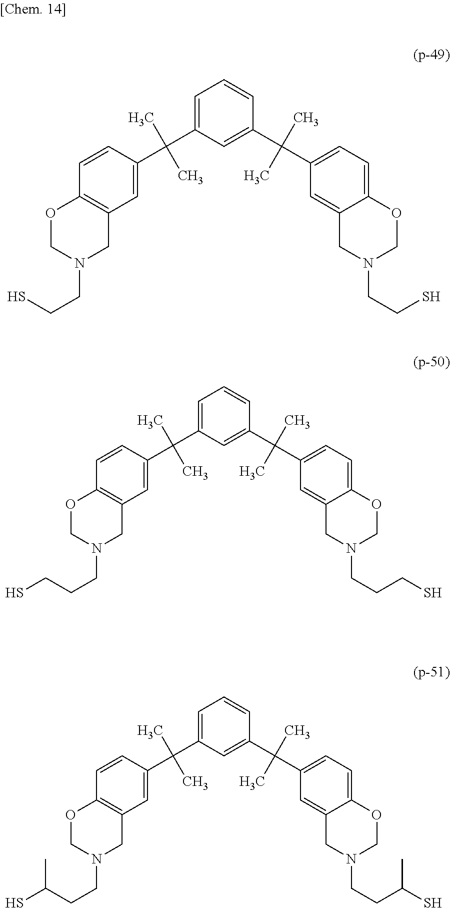 [SMILES: CC(C)(c1cccc(C(C)(C)c2ccc3c(c2)CN(CCS)CO3)c1)c1ccc2c(c1)CN(CCS)CO2 ; CC(C)(c1cccc(C(C)(C)c2ccc3c(c2)CN(CCCS)CO3)c1)c1ccc2c(c1)CN(CCCS)CO2 ; CC(S)CCN1COc2ccc(C(C)(C)c3cccc(C(C)(C)c4ccc5c(c4)CN(CCC(C)S)CO5)c3)cc2C1]

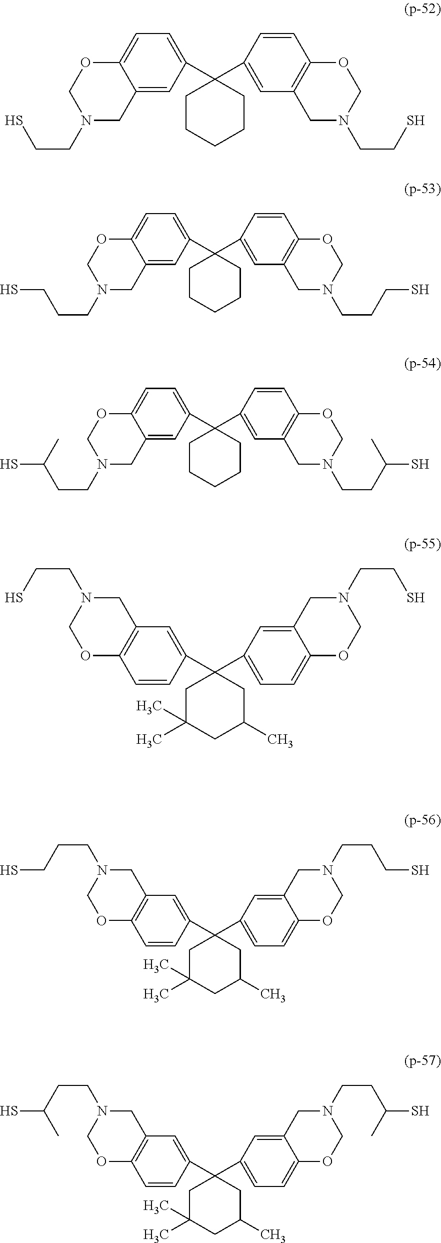 [SMILES: SCCN1COc2ccc(C3(c4ccc5c(c4)CN(CCS)CO5)CCCCC3)cc2C1 ; SCCCN1COc2ccc(C3(c4ccc5c(c4)CN(CCCS)CO5)CCCCC3)cc2C1 ; CC(S)CCN1COc2ccc(C3(c4ccc5c(c4)CN(CCC(C)S)CO5)CCCCC3)cc2C1 ; CC1CC(C)(C)CC(c2ccc3c(c2)CN(CCS)CO3)(c2ccc3c(c2)CN(CCS)CO3)C1 ; CC1CC(C)(C)CC(c2ccc3c(c2)CN(CCCS)CO3)(c2ccc3c(c2)CN(CCCS)CO3)C1 ; CC(S)CCN1COc2ccc(C3(c4ccc5c(c4)CN(CCC(C)S)CO5)CC(C)CC(C)(C)C3)cc2C1]

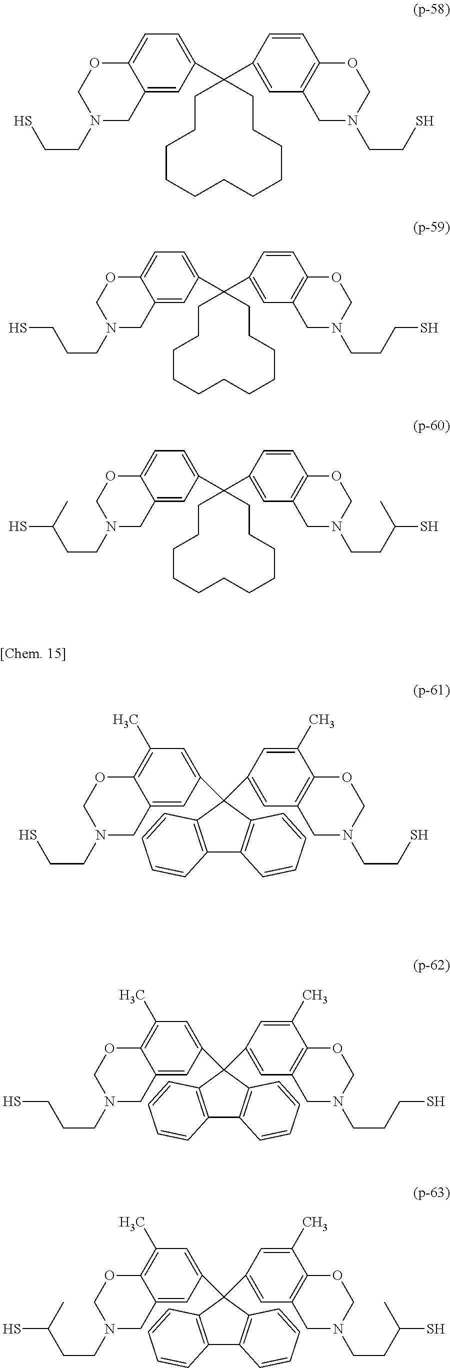 [SMILES: SCCN1COc2ccc(C3(c4ccc5c(c4)CN(CCS)CO5)CCCCCCCCCCC3)cc2C1 ; SCCCN1COc2ccc(C3(c4ccc5c(c4)CN(CCCS)CO5)CCCCCCCCCCC3)cc2C1 ; CC(S)CCN1COc2ccc(C3(c4ccc5c(c4)CN(CCC(C)S)CO5)CCCCCCCCCCC3)cc2C1 ; Cc1cc(C2(c3cc(C)c4c(c3)CN(CCS)CO4)c3ccccc3-c3ccccc32)cc2c1OCN(CCS)C2 ; Cc1cc(C2(c3cc(C)c4c(c3)CN(CCCS)CO4)c3ccccc3-c3ccccc32)cc2c1OCN(CCCS)C2 ; Cc1cc(C2(c3cc(C)c4c(c3)CN(CCC(C)S)CO4)c3ccccc3-c3ccccc32)cc2c1OCN(CCC(C)S)C2]

For the benzoxazine compound represented by general formula (1) according to the present invention, there are no particular limitations on the starting materials in the production of the benzoxazine compound and the method for producing the benzoxazine compound. For example, as illustrated by the following reaction formula, a production method in which a bisphenol compound represented by general formula (2), an aminothiol compound represented by general formula (3), and formaldehyde are allowed to undergo dehydration condensation reaction to cyclize, thereby obtaining the target benzoxazine compound represented by general formula (1), may be used.

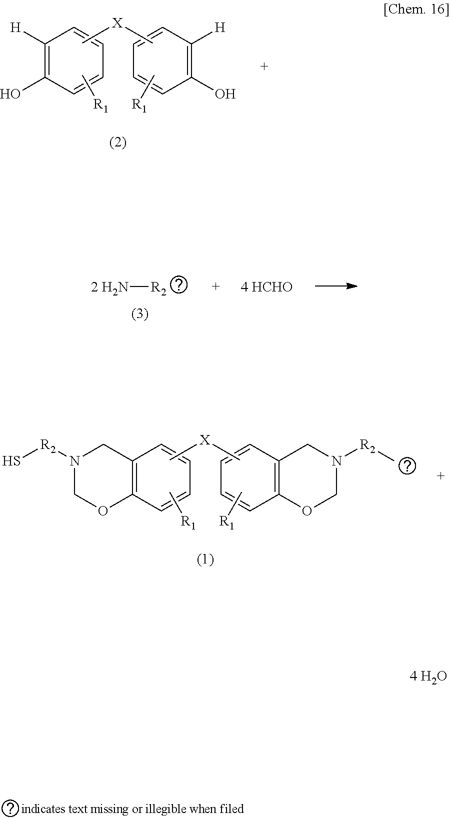

(In the formula, R₁, R₂, and X are as defined in general formula 1.)

In the above production method, a bisphenol compound represented by general formula (2), an aminothiol compound represented by general formula (3), and a formaldehyde are used as starting materials.

Specific examples of the bisphenol compound represented by general formula (2) include bisphenol F (bis(2-hydroxyphenyl) methane, 2-hydroxyphenyl-4-hydroxyphenylmethane, bis(4-hydroxyphenyl) methane), bisphenol E (1,1-bis(4-hydroxyphenyl) ethane), bisphenol A (2,2-bis(4-hydroxyphenyl) propane), bisphenol C (2,2-bis(4-hydroxy-3-methylphenyl) propane), 2,2-bis(4-hydroxyphenyl)-4-methylpentane, 4,4′-dihydroxybiphenyl, 4,4′-dihydroxy-3,3′-dimethylbiphenyl, bis(4-hydroxyphenyl) ether, 4,4′-dihydroxybenzophenone, bis(4-hydroxyphenyl) sulfone, bis(4-hydroxyphenyl) sulfide, 1,1-bis(4-hydroxyphenyl)-1-phenylethane, 1,1-bis(4-hydroxyphenyl)-1-naphthylethane, 2,2-bis(4-hydroxyphenyl) hexafluoropropane, bisphenol M (1,3-bis(2-(4-hydroxyphenyl)-2-propyl)benzene), bisphenol Z (1,1-bis(4-hydroxyphenyl)cyclohexane), bisphenol TMC (1,1-bis(4-hydroxyphenyl)-3,3,5-trimethylcyclohexane), 1,1-bis(4-hydroxyphenyl)cyclododecane, and 9,9-bis(4-hydroxy-3-methylphenyl) fluorene.

Specific examples of the aminothiol compound represented by general formula (3) include 2-aminoethanethiol, 3-amino-1-propanethiol, 2-amino-1-methylethanethiol, 2-amino-2-methylethanethiol, 5-amino-1-pentanethiol, and 6-amino-1-hexanethiol. Of these, 2-aminoethanethiol, 3-amino-1-propanethiol, 2-amino-1-methylethanethiol, 2-amino-2-methylethanethiol, 5-amino-1-pentanethiol, and 6-amino-1-hexanethiol are preferred, 2-aminoethanethiol, 3-amino-1-propanethiol, and 2-amino-1-methylethanethiol are more preferred, and 2-aminoethanethiol is particularly preferred.

Specific examples of the formaldehyde include an aqueous formaldehyde solution, 1,3,5-trioxane, and paraformaldehyde.

In the above production method, the amount of the formaldehyde used is preferably in the range of 4.0 to 20.0 mol, more preferably in the range of 4.0 to 16.0 mol, still more preferably in the range of 4.0 to 12.0 mol, relative to 1 mol of the bisphenol compound represented by general formula (2).

In the above production method, the amount of the aminothiol compound represented by general formula (3) used is preferably in the range of 2.0 to 10.0 mol, more preferably in the range of 2.0 to 8.0 mol, still more preferably in the range of 2.0 to 6.0 mol, relative to 1 mol of the bisphenol compound represented by general formula (2).

A catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

The reaction is typically performed in the presence of a solvent. The solvent is not particularly limited as long as it does not inhibit the reaction, and preferred examples include toluene, xylene, ethyl acetate, butyl acetate, chloroform, dichloromethane, tetrahydrofuran, and dioxane. These solvents can be used alone or in combination. The amount of solvent used is not particularly limited as long as the reaction is not hindered, and is typically in the range of 0.5 to 5 times, preferably in the range of 1 to 3 times the amount of the bisphenol compound represented by general formula (2) on a weight basis.

The reaction temperature is typically in the range of 10° C. to 150° C., preferably in the range of 10° C. to 120° C., more preferably in the range of 10° C. to 80° C., still more preferably in the range of 20° C. to 70° C., particularly preferably in the range of 20° C. to 60° C.

The reaction may be performed under normal pressure conditions, or may be performed under increased pressure or reduced pressure.

In another embodiment, a process of removing water derived from the raw materials or water generated during the reaction out of the system may be included. The process of removing water generated from a reaction solution is not particularly limited and can be performed by distilling the generated water azeotropically with the solvent system in the reaction solution. The generated water can be removed out of the reaction system by using, for example, an isobaric dropping funnel equipped with a cock, a Dimroth condenser, or a Dean-Stark apparatus.

From the final reaction mixture obtained, the benzoxazine compound represented by general formula (1) can be obtained by a known method after completion of the reaction. For example, deactivation treatment of the catalyst used, water washing treatment, and the like may be performed on the reaction mixture after the reaction, and the remaining raw materials and solvent may be distilled off from the reaction mixture to thereby obtain the target as a residual liquid. Other possible methods include adding the residual liquid to a poor solvent to obtain the target as a precipitate, and adding a solvent to the reaction mixture to cause crystallization and performing filtration to obtain the target as powder or particles. The benzoxazine compound collected by any of these methods can be made into a high-purity product by, for example, standard purification means such as washing with a solvent or water or recrystallization.

As the component (A) according to the present invention, two or more benzoxazine compounds represented by general formula (1) may be used in combination. Alternatively, two or more benzoxazine compounds represented by general formula (1) may be used in combination by using a mixture of the benzoxazine compounds represented by general formula (1) obtained by using two or more bisphenol compounds represented by general formula (2) in the reaction for producing the benzoxazine compounds represented by general formula (1). The ratio of the two or more bisphenol compounds represented by general formula (2) used in combination is not particularly limited.

Specifically, for example, when bisphenol F is used as the bisphenol compound represented by general formula (2), a mixture of positional isomers thereof, that is, bis(2-hydroxyphenyl) methane, 2-hydroxyphenyl-4-hydroxyphenylmethane, and bis(4-hydroxyphenyl) methane, can be used, and the ratio thereof is not particularly limited. Bisphenol F containing a large proportion of bis(2-hydroxyphenyl) methane can be obtained by, for example, the method of Japanese Unexamined Patent Application Publication No. 08-245464, and bisphenol F containing a large proportion of bis(4-hydroxyphenyl) methane can be obtained by, for example, the method of Japanese Unexamined Patent Application Publication No. 06-340565.

When such a mixture of positional isomers of bisphenol F and 2-aminoethanethiol as the aminothiol compound represented by general formula (3) are used to synthesize the benzoxazine compound represented by general formula (1) according to the present invention by the production method described above, a mixture of compounds (p-1), (p-4), and (p-7) can be obtained.

The bisphenol compound represented by general formula (2) for use may contain a polynuclear structure, which is a by-product formed during the production of a bisphenol (binuclear structure). The content ratio thereof is not particularly limited, and is preferably 50 wt % or less, more preferably 30 wt % or less, still more preferably 15 wt % or less.

Two or more benzoxazine compounds represented by general formula (1) may be used in combination by using a mixture of the benzoxazine compounds represented by general formula (1) obtained by using two or more aminothiol compounds represented by general formula (3) in the reaction for producing the benzoxazine compounds represented by general formula (1). The ratio of the two or more aminothiol compounds represented by general formula (3) used in combination is not particularly limited.

The benzoxazine compound represented by general formula (1) according to the present invention may be a crude product containing a compound produced as a by-product in the reaction for producing the benzoxazine compound. The compound produced as a by-product may be, for example, a compound having a molecular weight higher than that of the benzoxazine compound represented by general formula (1).

For the crude product of the benzoxazine compound represented by general formula (1), the content of the benzoxazine compound represented by general formula (1) is not particularly limited. The content can be analyzed by gel permeation chromatography using a differential refractometer as a detector. Typically, the lower limit of the peak area of the benzoxazine compound represented by general formula (1) relative to the area of all peaks detected in the analysis is 10 area % or more, preferably 20 area % or more, more preferably 30 area % or more, particularly preferably 40 area % or more. The upper limit thereof is 99.9 area %.

### <Component (B): Compound Having Three- or Four-Membered Ring Cyclic Ether Group>

The component (B) in the curable resin composition according to the present invention is a compound having a three- or four-membered ring cyclic ether group, and it is preferable to use a compound having a three-membered ring cyclic ether group.

Examples of the compound having a three-membered ring cyclic ether group include glycidyl ether compounds, alicyclic epoxy compounds, and epoxy resins, and these are preferred.

Specific examples of glycidyl ether compounds include glycidyl ether compounds obtained through the reaction of a polyhydric phenol with epichlorohydrin, such as bisphenol A diglycidyl ether (DGEBA), bisphenol F diglycidyl ether, bisphenol S diglycidyl ether, hexahydro bisphenol A diglycidyl ether, tetramethyl bisphenol A diglycidyl ether, resorcinol diglycidyl ether, biphenol diglycidyl ether, tetramethyl biphenol diglycidyl ether, hexamethyl biphenol diglycidyl ether, tetrabromo bisphenol A diglycidyl ether, and dihydroxynaphthalene diglycidyl ether.

Examples of alicyclic epoxy compounds include 3,4-epoxycyclohexylmethyl-3,4-epoxycyclohexane carboxylate, bi(3,4-epoxycyclohexyl), bis(3,4-epoxycyclohexyl) ether, bis(3,4-epoxycyclohexyl) methane, and 2,2-bis(3,4-epoxycyclohexyl) propane.

Examples of epoxy resins include phenol novolac epoxy resins, ortho-cresol epoxy resins, biphenyl epoxy resins, biphenyl aralkyl epoxy resins, naphthalene epoxy resins, anthracene dihydride epoxy resins, and brominated novolac epoxy resins.

As the compound having a four-membered ring cyclic ether group, for example, an oxetane compound can be used. Specific examples include 3-ethyl-3-hydroxymethyloxetane, 1,4-bis [(3-ethyl-3-oxetanyl) methoxymethyl]benzene, 3-ethyl-3-(phenoxymethyl) oxetane, di [(3-ethyl-3-oxetanyl)methyl]ether, 3-ethyl-3-[(2-ethylhexyloxymethyl)]oxetane, bis [(3-ethyl-3-oxetanyl)methyl]terephthalate, bis [(3-ethyl-3-oxetanyl)methyl]isophthalate, 4,4′-bis [(3-ethyl-3-oxetanyl) methoxymethyl]biphenyl, and phenol novolac oxetane.

### <Component (C): Compound Having Reactive Group Including Carbon-Carbon Double Bond or Carbon-Carbon Triple Bond>

The component (C) in the curable resin composition according to the present invention is a compound having a reactive group including a carbon-carbon double bond or a carbon-carbon triple bond.

Examples of the reactive group including a carbon-carbon double bond or a carbon-carbon triple bond include a vinyl group, a vinyl ether group, an allyl group, an allyl ether group, an acryloyl group, a methacryloyl group, a styrene group, a maleimide group, and alkynyl groups.

Of these, a compound having a maleimide group is preferred.

Examples of the compound having a maleimide group include bismaleimide compounds having the following structures, and specific examples include p-phenylene bismaleimide, m-phenylene bismaleimide, 4,4′-diphenylmethane bismaleimide, 4,4-diphenyl ether bismaleimide, 4,4-diphenyl sulfone bismaleimide, 2,2-bis [4-(4-maleimidophenoxy)phenyl]propane, and 1,3-bis(4-maleimidophenoxy)benzene.

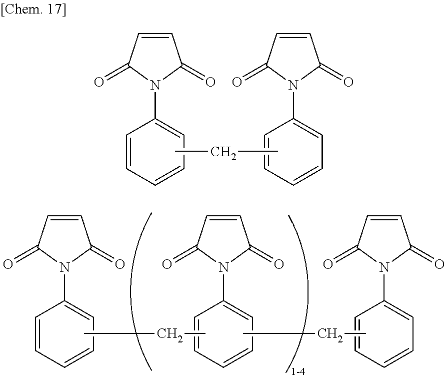

In the curable resin composition according to the present invention, the amount of the component (B) used, the amount of the component (C) used, or the total amount of the component (B) and the component (C) used is in the range of 5 to 2000 parts by weight relative to 100 parts by weight of the component (A). It is preferably in the range of 10 to 1000 parts by weight relative to 100 parts by weight of the component (A), more preferably in the range of 20 to 500 parts by weight relative to 100 parts by weight of the component (A), particularly preferably in the range of 50 to 200 parts by weight relative to 100 parts by weight of the component (A).

### <Component (D): Curing Reaction Catalyst>

The curable resin composition according to the present invention can contain a curing reaction catalyst as a component (D).

Examples of curing reaction catalysts that can be used include acid catalysts, alkaline catalysts, and phosphorus compounds. Of these, acid catalysts are preferred.

The acid catalyst is preferably an organic acid catalyst, and examples of organic acid catalysts include para-toluenesulfonic acid and methanesulfonic acid.

The alkaline catalyst is preferably an organic alkaline catalyst, and examples of organic alkaline catalysts include tertiary amines such as 1,8-diaza-bicyclo [5.4.0]undec-7-ene, triethylenediamine, and tris(2,4,6-dimethylaminomethyl) phenol, and imidazoles such as 2-ethyl-4-methylimidazole and 2-methylimidazole.

Examples of phosphorus compounds include triphenylphosphine, tetraphenylphosphonium bromide, tetraphenylphosphonium tetraphenylborate, and tetra-n-butylphosphonium-O,O-diethyl phosphorodithioate.

Among them, para-toluenesulfonic acid, 2-methylimidazole, and triphenylphosphine are particularly preferred. These may be used alone or in combination.

The amount of the component (D) used is in the range of 0.1 wt % to 20 wt % relative to the total amount of the component (A), the component (B), and the component (C) used. It is preferably in the range of 0.1 wt % to 15 wt %, more preferably in the range of 0.1 wt % to 10 wt %, particularly preferably in the range of 0.1 wt % to 8 wt %.

### <Component (E): Filler>

The curable resin composition according to the present invention can contain a filler as a component (E).

The filler as the component (E) may be silicon oxide, aluminum oxide, magnesium oxide, boron nitride, aluminum nitride, silicon nitride, or silicon carbide, and can be used as a mixture with an inorganic filler such as hexagonal boron nitride and a reinforcement fiber such as carbon fiber, glass fiber, organic fiber, boron fiber, steel fiber, or aramid fiber.

The curable resin composition according to the present invention may contain a curable resin material other than the components (A) to (E) above, and examples of such a material include phenol resins and benzoxazine compounds other than the benzoxazine compound represented by general formula (1).

Examples of phenol resins include novolac phenol resins such as phenol novolac resin, cresol novolac resin, naphthol novolac resin, aminotriazine novolac resin, and trisphenylmethane phenol novolac resin; modified phenol resins such as terpene-modified phenol resin and dicyclopentadiene-modified phenol resin; aralkyl resins such as phenol aralkyl resins having a phenylene backbone and/or a biphenylene backbone and naphthol aralkyl resins having a phenylene backbone and/or a biphenylene backbone; and resol phenol resins.

Examples of benzoxazine compounds other than the benzoxazine compound represented by general formula (1) include benzoxazine compounds having structures represented by general formulae (A) to (C) below.

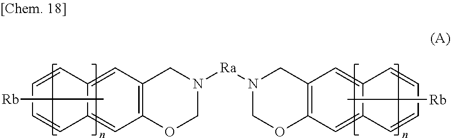

(In the formula, Ra represents a divalent group having 1 to 30 carbon atoms, each Rb independently represents an optionally substituted monovalent group having 1 to 10 carbon atoms, and n represents 0 or 1.)

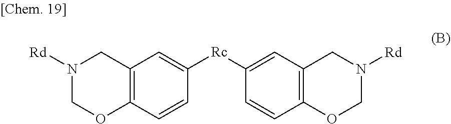

(In the formula, Rc represents a divalent group having 1 to 30 carbon atoms, a direct bond, an oxygen atom, a sulfur atom, a carbonyl group, or a sulfonyl group, and each Rd independently represents a monovalent group having 1 to 10 carbon atoms.)

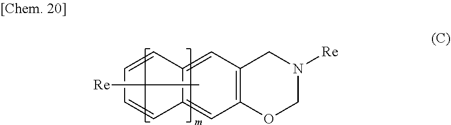

(In the formula, each Re independently represents a monovalent group having 1 to 10 carbon atoms, and m represents 0 or 1.)

Ra in the benzoxazine compound having the structure represented by general formula (A) represents a divalent group having 1 to 30 carbon atoms. Specific examples thereof include alkylene groups such as 1,2-ethylene, 1,4-butylene, and 1,6-hexylene; alkylene groups including a cyclic structure, such as 1,4-cyclohexylene, dicyclopentadienylene, and adamantylene; and arylene groups such as 1,4-phenylene, 4,4′-biphenylene, diphenyl ether-4,4′-diyl, diphenyl ether-3,4′-diyl, diphenyl ketone-4,4′-diyl, and diphenyl sulfone-4,4′-diyl.

Each Rb in the benzoxazine compound having the structure represented by general formula (A) independently represents a monovalent group having 1 to 10 carbon atoms. Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these groups may further have a substituent such as an alkoxy group having 1 to 4 carbon atoms, an acyl group having 1 to 4 carbon atoms, a halogen atom, a carboxyl group, a sulfo group, an allyloxy group, a hydroxy group, or a thiol group.

Examples of the benzoxazine compound having the structure represented by general formula (A) include P-d type benzoxazine manufactured by Shikoku Chemicals Corporation, and JBZ-OP100N and JBZ-BP100N manufactured by JFE Chemical Corporation.

Rc in the benzoxazine compound having the structure represented by general formula (B) represents a divalent group having 1 to 30 carbon atoms, a direct bond, an oxygen atom, a sulfur atom, a carbonyl group, or a sulfonyl group. Examples of the divalent group having 1 to 30 carbon atoms include alkylene groups such as methylene, 1,2-ethylene, 1,4-butylene, and 1,6-hexylene; alkylene groups including a cyclic structure, such as 1,4-cyclohexylene, dicyclopentadienylene, and adamantylene; and alkylidene groups such as ethylidene, propylidene, isopropylidene, butylidene, phenylethylidene, cyclopentylidene, cyclohexylidene, cycloheptylidene, cyclododecylidene, 3,3,5-trimethylcyclohexylidene, and fluorenylidene.

Each Rd in the benzoxazine compound having the structure represented by general formula (B) independently represents a monovalent group having 1 to 10 carbon atoms. Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these substituents may further have a substituent such as an alkoxy group having 1 to 4 carbon atoms, an acyl group having 1 to 4 carbon atoms, a halogen atom, a carboxyl group, a sulfo group, an allyloxy group, or a hydroxy group.

Examples of the benzoxazine compound having the structure represented by general formula (B) include F-a type benzoxazine manufactured by Shikoku Chemicals Corporation and BS-BXZ manufactured by Konishi Chemical Ind. Co., Ltd.

Each Re in the benzoxazine compound having the structure represented by general formula (C) independently represents a monovalent group having 1 to 10 carbon atoms.

Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these substituents may further have a substituent such as an alkoxy group having 1 to 4 carbon atoms, an acyl group having 1 to 4 carbon atoms, a halogen atom, a carboxyl group, a sulfo group, an allyloxy group, a hydroxy group, or a thiol group.

The curable resin composition according to the present invention may contain a solvent as a component (F). In particular, the curable resin composition is preferably dissolved or dispersed in the component (F) to be in the form of a varnish.

The component (F) is not particularly limited as long as it dissolves or disperses the curable resin composition according to the present invention, and for example, an aromatic hydrocarbon solvent, an aliphatic ketone solvent having 3 to 7 carbon atoms, or an ether solvent can be used.

The usable amount of the solvent is not limited as long as, for example, each component can be sufficiently dissolved or dispersed, and is 10 times or less, more preferably 5 times or less, still more preferably 1 times or less, particularly preferably 0.5 times or less, on a weight basis, the total amount of the component (A), and the component (B) and/or the component (C) used.

The varnish can be used, for example, for forming a film-like resin composition by applying the varnish onto a support using a coater and further drying the varnish, or for producing a composition by impregnating a reinforcing fiber with the varnish and then removing the solvent.

The curable resin composition according to the present invention is obtained by mixing the benzoxazine compound represented by general formula (1), that is, the component (A), at least one of the component (B) and the component (C), and optionally the components (D) to (F) and other curable resin materials. The method of the mixing is not particularly limited, and a method known in the art can be employed depending on the components used. Examples of the method include mixing using a mixer or the like and melt mixing using a kneader or the like. The mixing of the components may be performed either in air or in an inert gas atmosphere such as nitrogen, but is preferably performed in an inert gas atmosphere in order to prevent oxygen-induced degradation.

The curable resin composition according to the present invention may entrain bubbles when cured if water or a residual solvent is contained in the composition, and thus to prevent this, it is preferable to perform a vacuum degassing treatment as a pretreatment. The vacuum degassing treatment may be performed at any temperature at which the curable resin composition according to the present invention is in a molten state but is preferably performed at up to 150° C. because curing does not proceed and degassing is facilitated. The vacuum degassing treatment is preferably, but not necessarily, performed at a low pressure (highly reduced pressure) and may be performed either in air or in an inert gas atmosphere such as nitrogen. The vacuum degassing treatment is preferably performed until no bubbles can be visually observed.

### <Cured Product Obtained by Curing Curable Resin Composition According to Present Invention>

A cured product according to the present invention can be obtained by curing the curable resin composition according to the present invention.

A method for producing the cured product according to the present invention may have a curing step of performing a reaction of curing the curable resin composition under a high-temperature condition. The method may have, before the curing step, a pre-curing step of performing a curing reaction at a temperature lower than that in the curing step, and preferably has the pre-curing step.

The temperature condition in the pre-curing step is in the range of 60° C. or higher and lower than 150° C., preferably in the range of 70° C. to 140° C., more preferably in the range of 80° C. to 130° C., particularly preferably in the range of 90° C. to 130° C.

The temperature condition in the curing step is in the range of 150° C. to 240° C., preferably in the range of 150° C. to 220° C., more preferably in the range of 150° C. to 210° C., particularly preferably in the range of 150° C. to 200° C.

When curing is performed in such a temperature range, the reaction time may be about 1 to 10 hours.

The curing step and the pre-curing step may be performed either in air or in an inert gas atmosphere such as nitrogen, but is preferably performed in an inert gas atmosphere in order to prevent oxygen-induced degradation of the cured product obtained.

The curable resin composition according to the present invention can suppress the generation of an odorous volatile component during the production of a cured product.

In addition, the curable resin composition according to the present invention can provide a cured product having significantly improved heat resistance as compared with the case where only the benzoxazine compound having a thiol group is used.

It has been shown that the benzoxazine compound having a thiol group in the curable resin composition according to the present invention invented by the present inventors has a lower curing temperature than benzoxazine compounds known in the art and thus can improve working efficiency through reduced time and saved energy during curing and can be used also for a heat-sensitive material (substrate), and furthermore that the cured product thereof can melt at a lower temperature than benzoxazine compounds having a hydroxy group, and thus the curable resin composition obtained using the benzoxazine compound can be produced and handled at a low temperature.

In view of the foregoing, the curable resin composition according to the present invention and the cured product obtained therefrom can be used as useful materials in the fields of prepregs, printed circuit boards, sealants for electronic components, electrical and electronic molded parts, insulating substrates, liquid crystal alignment agents, semiconductor sealing materials, automotive parts, laminated materials, paints, resist inks, and the like.

### EXAMPLES

The present invention will now be described more specifically with reference to Examples.

### <Analysis Method>

### 1. Analysis of Reaction Solution Composition and Purity of Benzoxazine Compound (Gel Permeation Chromatography: GPC)

The purity of a benzoxazine compound synthesized was defined as the area percentage of the benzoxazine compound determined by this analysis.

Apparatus: HLC-8320/manufactured by Tosoh Corporation

Detector: differential refractometer (RI)

### [Measurement Conditions]

Flow rate: 1 mL/min

Eluate: tetrahydrofuran

Temperature: 40° C.

Wavelength: 254 nm

Measurement sample: A solution obtained by diluting 1 g of a benzoxazine compound-containing composition 200 times with tetrahydrofuran was used as a measurement sample.

### 2. Measurement of Weight Loss Rate of Curable Resin Composition During Curing

Using a mortar, 5 g of a component (A) (a benzoxazine compound), 5 g of a component (B) (a compound having a three- or four-membered ring cyclic ether group) and/or a component (C) (a compound having a reactive group including a carbon-carbon double bond or a carbon-carbon triple bond), and a component (D) (a curing reaction catalyst) in an amount of 5 wt % relative to the total amount of the component (A) and the component (B) and/or the component (C) loaded were pulverized and mixed to prepare a curable resin composition.

The composition was loaded in a 50 mL test tube and then heated in a nitrogen atmosphere under predetermined temperature and time conditions described in Examples and Comparative Examples, and the weights of the mixture before and after heating were measured. A value calculated by dividing the weight difference by the weight of the mixture before heating was used as a weight loss rate.

### 3. Measurement of Amount of Sulfur-Containing Volatile Component Generated During Curing of Curable Resin Composition

The amount of sulfur-containing volatile component generated was calculated by analyzing a sulfur-containing volatile component with the following apparatuses under the following conditions and using a calibration curve method. Analyzer: GC-2010 Plus/manufactured by Shimadzu Corporation Vaporizer: TurboMatrix 40/manufactured by PerkinElmer, Inc.

### [Measurement Conditions]

Vaporization chamber temperature: 300° C.

Carrier gas: nitrogen

Total flow rate: 50.0 mL/min

Column flow rate: 0.74 mL/min

Column: TC-1

Vaporization pressure: 240 kPa

Holding temperature/temperature holding time: described in Examples and Comparative Examples

Pressing time: 3.0 min

Injection time: 0.10 min

Measurement sample: Using a mortar, 2 g of a component (A), 2 g of a component (B) and/or a component (C), and a component (D) in an amount of 5 wt % relative to the total amount of the component (A) and the component (B) or the component (C) loaded were pulverized and mixed to prepare a curable resin composition. The mixture was then placed in an HS-GC vial. The vial was brought under a nitrogen atmosphere and then sealed with an aluminum cap.

The sealed HS-GC vial was heated at the above holding temperature for the above temperature holding time, and the vapor-phase portion in the HS-GC vial was then analyzed.

### 4. Production of Cured Product

The production of a cured product was performed using a constant-temperature drying oven.

Apparatus: vacuum constant-temperature drying oven DP-32/manufactured by Yamato Scientific Co., Ltd.

Production vessel: silicone casting plate for DMA measurement

### 5. Measurement of Glass Transition Temperature (Tg) of Cured Product

Apparatus: Discovery DMA850/manufactured by TA Instruments

### [Measurement Conditions]

Measurement mode: three-point bending

Heating rate: 2° C./min.

Fundamental frequency: 1 Hz

Atmosphere: in air stream

Measurement sample size: 50×8×3 mm

### <Synthesis Example 1> (Synthesis of Benzoxazine Compound a Represented by Chemical Formula Below)

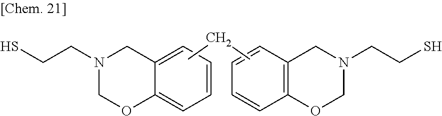

In a 500 mL four-necked flask equipped with a thermometer, a stirrer, a condenser, and a dropping funnel, 31 g (0.15 mol) of bisphenol F (binuclear structure content, 90.1 wt %; isomer ratio thereof: bis(2-hydroxyphenyl) methane, 18.8 wt %; 2-hydroxyphenyl-4-hydroxyphenylmethane, 49.3 wt %; and bis(4-hydroxyphenyl) methane, 31.9 wt %; polynuclear structure content, 9.9 wt %), 74 g of 94% paraformaldehyde, and 57 g of toluene were loaded. After the reaction vessel was purged with nitrogen, the temperature of the mixed solution was adjusted to 30° C. Thereafter, 24 g of 2-aminoethanethiol was added dropwise into the four-necked flask using a dropping funnel over 1 hour while maintaining the temperature at 30° C. After completion of the dropwise addition, stirring was further performed at 30° C. for 3 hours. The composition of the reaction solution was analyzed by GPC according to the above analysis method, revealing that the percentage of the above target compound present in the reaction solution was 88 area %. After completion of the reaction, alkali washing was performed using a 3% aqueous sodium hydroxide solution, and water washing was then performed until the pH of the reaction solution became 7 or less. Thereafter, toluene and water were removed by reduced-pressure distillation at 30° C. The pressure during the distillation was gradually reduced so as to finally reach 2.3 kPa. After the solvent was removed to some extent, the residual solvent was further removed at 90° C. and 2.8 kPa. The composition containing the target compound was taken out, solidified by cooling, and then pulverized to obtain 156 g of the target compound (purity: 75%, compounds with molecular weights higher than that of the target compound: 25 area %).

The results of 1H-NMR analysis confirmed that the target compound represented by the above chemical formula was obtained.

¹H-NMR analysis (400 MHZ, solvent: CDCl3, reference material: tetramethylsilane): 1.32-1.95 (2H, brm), 2.91-3.05 (4H, m), 3.07-3.22 (4H, m), 3.64-4.13 (10H, m), 6.66-7.12 (6H, m).

### Comparative Example 1

Under the same conditions as the above conditions for the measurement of a weight loss rate during curing and the measurement of the amount of sulfur-containing volatile component generated, except that only the benzoxazine compound A obtained in Synthesis Example 1 was used as the component (A), and the component (B) and/or the component (C) and the component (D) were not used, the weight loss rate during curing and the amount of sulfur-containing volatile component generated were measured. Heating was performed at a temperature of 175° C. for 1 hour.

The sulfur-containing volatile component in the case where the benzoxazine compound A was used was confirmed to be thiazolidine. Presumably, thiazolidine was generated through the process represented by the following formula.

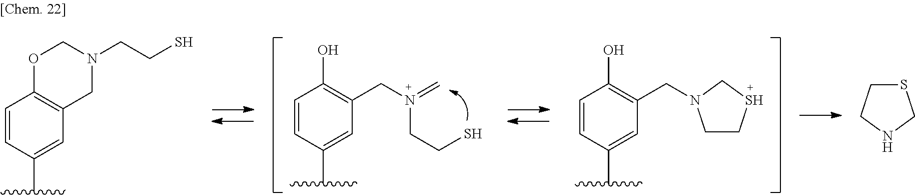

As a result, the weight loss rate was 2.3 wt %, and the amount of thiazolidine generated was 36.2 mol %.

The results of Comparative Example 1 have shown that the curable resin composition obtained using only the component (A), that is, the benzoxazine compound A having a thiol group, experiences a weight loss during the production of a cured product and generation of a great amount of sulfur-containing volatile component (thiazolidine).

### Example 1

Under the above conditions for the measurement of the amount of sulfur-containing volatile component generated, the amount of sulfur-containing volatile component (thiazolidine) generated in the case of using the benzoxazine compound A obtained in Synthesis Example 1 as the component (A) and 4,4′-diphenylmethane bismaleimide (BMI) as the component (C) and not using the component (D) was measured. Heating was performed at a temperature of 175° C. for 1 hour.

As a result, the amount of thiazolidine generated was 17.1 mol %.

### Example 2

Under the above conditions for the measurement of the amount of sulfur-containing volatile component generated, the amount of sulfur-containing volatile component (thiazolidine) generated in the case of using the benzoxazine compound A obtained in Synthesis Example 1 as the component (A) and bisphenol A diglycidyl ether (DGEBA) as the component (B) and not using the component (D) was measured. Heating was performed at a temperature of 175° C. for 1 hour.

As a result, it was confirmed that thiazolidine was not generated.

The results of Examples 1 and 2 have shown that the curable resin composition curing agent according to the present invention containing the component (B) and/or the component (C) in addition to the benzoxazine compound having a thiol group can suppress the generation of a sulfur-containing volatile component (thiazolidine).

### Comparative Example 2

Under the same conditions as the above conditions for the measurement of a weight loss rate during curing and the measurement of the amount of sulfur-containing volatile component generated, except that only the benzoxazine compound A obtained in Synthesis Example 1 was used as the component (A), and the component (B) and/or the component (C) and the catalyst were not used, the weight loss rate during curing and the amount of sulfur-containing volatile component (thiazolidine) generated were measured. After heating was performed at a temperature of 120° C. for 1 hour, heating was performed at a temperature of 175° C. for 4 hours.

As a result, the weight loss rate was 3.6 wt %, and the amount of thiazolidine generated was 56.0 mol %.

### Example 3

Under the above conditions for the measurement of the amount of sulfur-containing volatile component generated, the amount of sulfur-containing volatile component (thiazolidine) generated in the case of using the benzoxazine compound A obtained in Synthesis Example 1 as the component (A) and 4,4′-diphenylmethane bismaleimide (BMI) as the component (C) and not using the component (D) was measured. After heating was performed at a temperature of 120° C. for 1 hour, heating was performed at a temperature of 175° C. for 4 hours.

As a result, the amount of thiazolidine generated was 3.0 mol %.

### Example 4

Under the above conditions for the measurement of a weight loss rate during curing and the measurement of the amount of sulfur-containing volatile component generated, the weight loss rate and the amount of sulfur-containing volatile component (thiazolidine) generated in the case of using the benzoxazine compound A obtained in Synthesis Example 1 as the component (A) and bisphenol A diglycidyl ether (DGEBA) as the component (B) and not using the component (D) were measured. After heating was performed at a temperature of 120° C. for 1 hour, heating was performed at a temperature of 175° C. for 4 hours.

As a result, the weight loss rate was 0.9 wt %. It was confirmed that thiazolidine was not generated.

The results of Examples 3 and 4 have shown that the curable resin composition according to the present invention containing the benzoxazine compound having a thiol group, that is, the component (A), and the component (B) and/or the component (C) can further suppress the generation of thiazolidine when a pre-curing step at 120° C. is performed in the curing reaction.

By contrast, the results of Comparative Example 2 have shown that the curable resin composition obtained using only the benzoxazine compound having a thiol group, although under the curing reaction conditions where the generation of thiazolidine can be suppressed in Examples 3 and 4, cannot suppress the generation of thiazolidine because of not containing the component (B) or the component (C).

### Example 5

The amount of sulfur-containing volatile component (thiazolidine) generated in the case of using the benzoxazine compound A obtained in Synthesis Example 1 as the component (A), BMI as the component (C), and 2-methylimidazole (2 MI) as the component (D) was measured. Heating was performed at a temperature of 175° C. for 1 hour.

As a result, the amount of thiazolidine generated was 23.8 mol %.

### Example 6

The amount of sulfur-containing volatile component (thiazolidine) generated in the case of using the benzoxazine compound A obtained in Synthesis Example 1 as the component (A), BMI as the component (C), and 2-methylimidazole (2 MI) as the component (D) was measured. After heating was performed at a temperature of 120° C. for 1 hour, heating was performed at a temperature of 175° C. for 4 hours.

As a result, the amount of thiazolidine generated was 14.2 mol %.

### Example 7

The amount of sulfur-containing volatile component (thiazolidine) generated in the case of using the benzoxazine compound A obtained in Synthesis Example 1 as the component (A), DGEBA as the component (B), and 2-methylimidazole (2 MI) as the component (D) was measured. After heating was performed at a temperature of 120° C. for 1 hour, heating was performed at a temperature of 175° C. for 4 hours.

As a result, the amount of thiazolidine generated was 34.6 mol %.

### Example 8

The amount of sulfur-containing volatile component (thiazolidine) generated in the case of using the benzoxazine compound A obtained in Synthesis Example 1 as the component (A), DGEBA as the component (B), and triphenylphosphine (TPP) as the component (D) was measured. Heating was performed at a temperature of 175° C. for 1 hour.

As a result, the amount of thiazolidine generated was 11.2 mol %.

### Example 9

The weight loss rate and the amount of sulfur-containing volatile component (thiazolidine) generated in the case of using the benzoxazine compound A obtained in Synthesis Example 1 as the component (A), DGEBA as the component (B), and triphenylphosphine (TPP) as the component (D) were measured. After heating was performed at a temperature of 120° C. for 1 hour, heating was performed at a temperature of 175° C. for 4 hours.

As a result, the weight loss rate was 1.0 wt %, and the amount of thiazolidine generated was 0.6 mol %.

### Example 10

The amount of sulfur-containing volatile component (thiazolidine) generated in the case of using the benzoxazine compound A obtained in Synthesis Example 1 as the component (A), BMI as the component (C), and triphenylphosphine (TPP) as the component (D) was measured. After heating was performed at a temperature of 120° C. for 1 hour, heating was performed at a temperature of 175° C. for 4 hours.

As a result, the amount of thiazolidine generated was 11.0 mol %.

### Example 11

The amount of sulfur-containing volatile component (thiazolidine) generated in the case of using the benzoxazine compound A obtained in Synthesis Example 1 as the component (A), BMI as the component (C), and para-toluenesulfonic acid monohydrate (PTSA) as the component (D) was measured. Heating was performed at a temperature of 175° C. for 1 hour.

As a result, the amount of thiazolidine generated was 4.6 mol %.

### Example 12

The weight loss rate and the amount of sulfur-containing volatile component (thiazolidine) generated in the case of using the benzoxazine compound A obtained in Synthesis Example 1 as the component (A), BMI as the component (C), and para-toluenesulfonic acid monohydrate (PTSA) as the component (D) were measured. After heating was performed at a temperature of 120° C. for 1 hour, heating was performed at a temperature of 175° C. for 4 hours.

As a result, the weight loss rate was 2.1 wt %, and the amount of thiazolidine generated was 1.0 mol %.

### Example 13

The weight loss rate and the amount of sulfur-containing volatile component (thiazolidine) generated in the case of using the benzoxazine compound A obtained in Synthesis Example 1 as the component (A), DGEBA as the component (B), and para-toluenesulfonic acid monohydrate (PTSA) as the component (D) were measured. After heating was performed at a temperature of 120° C. for 1 hour, heating was performed at a temperature of 175° C. for 4 hours.

As a result, the weight loss rate was 1.5 wt %. It was confirmed that thiazolidine was not generated.

It has been shown that the curable resin composition according to the present invention containing the benzoxazine compound having a thiol group, that is, the component (A), and the component (B) and/or the component (C), even when further containing a curing reaction catalyst as the component (D), can suppress the generation of a sulfur-containing volatile component (thiazolidine) as compared with the case where a cured product formed only of the benzoxazine compound having a thiol group is produced.

It has been shown that when PTSA, which is an acid catalyst, is used as the component (D), the generation of thiazolidine can be further suppressed.

The measurement results of the weight loss rate during curing and the amount (mol %) of sulfur-containing volatile component (thiazolidine) generated of the curable resin compositions of Examples 1 to 13 and Comparative Examples 1 and 2 and the sulfur-containing volatile component suppression rate (%) of Examples 1 to 13 calculated compared with Comparative Examples in which curing was performed under the same curing conditions using only the component A are listed in Table 1 below. In the table, the “Temperature/time in curing” column represents the temperature and time conditions in curing; specifically, (i) means conditions where heating is performed at a temperature of 175° C. for 1 hour, and (ii) means conditions where heating is performed at a temperature of 120° C. for 1 hour, and then heating is performed at a temperature of 175° C. for 4 hours. In the weight loss rate column, “-” represents being not measured.

TABLE 1

| | Curable | | | Temper- | Weight | Sulfur-containing | | |
|---|---|---|---|---|---|---|---|---|
| | resin composition | | | ature/ | loss | volatile component | | |
| | Com- | Com- | Com- | time | rate | Amount | | Comparative |
| | ponent | ponent | ponent | in | (wt | generated | Suppression | Example to |
| | B | C | D | curing | %) | (mol %) | rate (%) | be compared |
| Comparative | — | — | — | (i) | 2.3 | 36.2 | — | — |
| Example 1 | | | | | | | | |
| Example 1 | — | BMI | — | (i) | — | 17.1 | 52.8 | Comparative |
| | | | | | | | | Example 1 |
| Example 2 | DGEBA | — | — | (i) | — | not | 100.0 | Comparative |
| | | | | | | generated | | Example 1 |
| Comparative | — | — | — | (ii) | 3.6 | 56.0 | — | — |
| Example 2 | | | | | | | | |
| Example 3 | — | BMI | — | (ii) | — | 3.0 | 82.5 | Comparative |
| | | | | | | | | Example 2 |
| Example 4 | DGEBA | — | — | (ii) | 0.9 | not | 100.0 | Comparative |
| | | | | | | generated | | Example 2 |
| Example 5 | — | BMI | 2MI | (i) | — | 23.8 | 34.3 | Comparative |
| | | | | | | | | Example 1 |
| Example 6 | — | BMI | 2MI | (ii) | — | 14.2 | 74.6 | Comparative |
| | | | | | | | | Example 2 |
| Example 7 | DGEBA | — | 2MI | (ii) | — | 34.6 | 38.2 | Comparative |
| | | | | | | | | Example 2 |
| Example 8 | DGEBA | — | TPP | (i) | — | 11.2 | 80.0 | Comparative |
| | | | | | | | | Example 1 |
| Example 9 | DGEBA | — | TPP | (ii) | 1.0 | 0.6 | 98.9 | Comparative |
| | | | | | | | | Example 2 |
| Example 10 | — | BMI | TPP | (ii) | — | 11.0 | 80.4 | Comparative |
| | | | | | | | | Example 2 |
| Example 11 | — | BMI | PTSA | (i) | — | 4.6 | 87.3 | Comparative |
| | | | | | | | | Example 1 |
| Example 12 | — | BMI | PTSA | (ii) | 2.1 | 1.0 | 98.2 | Comparative |
| | | | | | | | | Example 2 |
| Example 13 | DGEBA | — | PTSA | (ii) | 1.5 | not | 100.0 | Comparative |
| | | | | | | generated | | Example 2 |

(Evaluation of heat resistance of cured product)

### Example 14

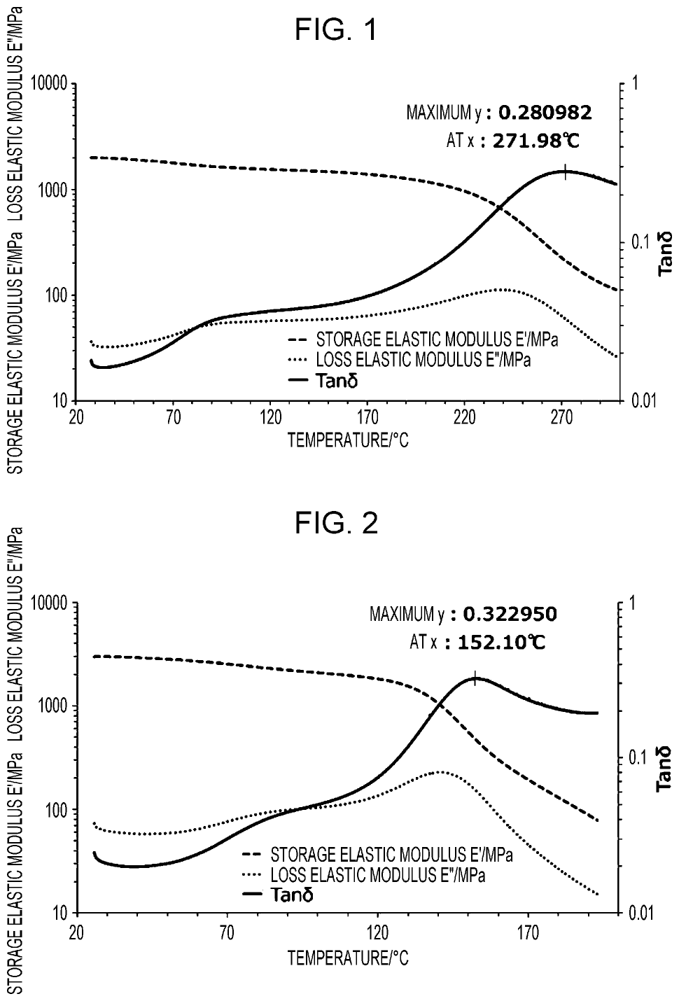

Using a mortar, 8 g of the benzoxasine compound A obtained in Synthesis Example 1, that is, the component (A), and 8 g of BMI, that is, the component (C), were pulverized and mixed. The mixture was melted and degassed at 120° C. for 3 hours and then cast into a preheated silicone casting plate for DMA measurement. Thereafter, the resulting product was cured by heating in a dryer under the conditions of 140° C.→150° C.→160° C.→180° C.→200° C.→220° C.→240° C./each for 2 hours and cooled overnight to obtain a cured product. The cured product obtained was subjected to dynamic viscoelasticity measurement, and Tg was calculated from the value of Tan δ and found to be 272° C. A chart of dynamic viscoelasticity analysis (DMA) of the cured product obtained is shown in FIG. 1.

### Comparative Example 3

Using a mortar, 9 g of the benzoxazine compound A obtained in Synthesis Example 1, that is, the component (A), was pulverized. The resulting product was melted and degassed at 100° C. for 1.5 hours and then cast into a preheated silicone casting plate for DMA measurement. Thereafter, the resulting product was cured by heating in a dryer under the conditions of 140° C.→150° C.→160° C.→180° C.→200° C./each for 2 hours and cooled overnight to obtain a cured product. The cured product obtained was subjected to dynamic viscoelasticity measurement, and Tg was calculated from the value of Tan δ and found to be 152° C. A chart of dynamic viscoelasticity analysis (DMA) of the cured product obtained is shown in FIG. 2.

The results of Example 14 and Comparative Example 3 have shown that the cured product obtained from the curable resin composition according to the present invention containing the benzoxazine compound having a thiol group and a curing agent including the component (B) and/or the component (C) has significantly improved heat resistance as compared with the cured product obtained using only the benzoxazine compound having a thiol group.

1. A curable resin composition comprising 100 parts by weight of a component (A) below and 5 to 2000 parts by weight of at least one of a component (B) below and a component (C) below:
(A): a benzoxazine compound represented by general formula (1) below: 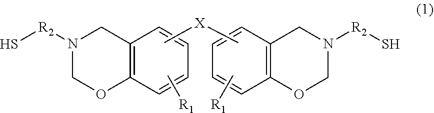
in the formula, each R₁ independently represents a hydrogen atom or an alkyl group having 1 to 6 carbon atoms, each R₂ independently represents an alkylene group having 1 to 10 carbon atoms including a linear, branched, or aliphatic-ring-containing alkylene group, and X represents a single bond, an oxygen atom, a sulfur atom, a sulfonyl group, a carbonyl group, or a divalent group represented by formula (1a) or formula (1b) below: 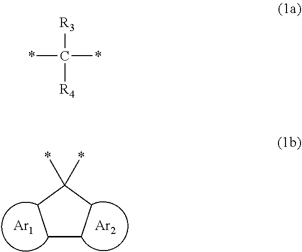
in formula (1a) and formula (1b), R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position;
(B): a compound having a three- or four-membered ring cyclic ether group;
(C): a compound having a reactive group including a carbon-carbon double bond or a carbon-carbon triple bond.

2. The curable resin composition according to claim 1, further comprising a component (D) below:
(D): a curing reaction catalyst.

3. The curable resin composition according to claim 2, wherein the curing reaction catalyst is an acid catalyst.

4. The curable resin composition according to claim 1, further comprising a component (E) below:
(E): a filler.

5. A varnish comprising the curable resin composition according to claim 1 and a component (F) below:
(F): an organic solvent.

6. A cured product obtained by curing the curable resin composition according to claim 1.

7. A method for producing a cured product containing a component (A) below, characterized by curing a curable resin composition containing the component (A) and at least one of a component (B) and a component (C) below:
(A): a benzoxazine compound represented by general formula (1) below: 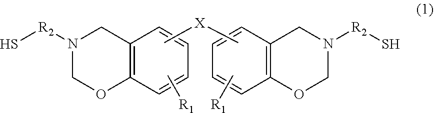
in the formula, each R₁ independently represents a hydrogen atom or an alkyl group having 1 to 6 carbon atoms, each R₂ independently represents a linear, branched, or aliphatic ring-containing an alkylene group having 1 to 10 carbon atoms including a linear, branched, or aliphatic-ring-containing alkylene group, and X represents a single bond, an oxygen atom, a sulfur atom, a sulfonyl group, a carbonyl group, or a divalent group represented by formula (1a) or formula (1b) below: 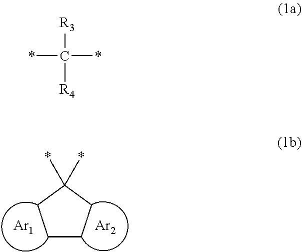
in formula (1a) and formula (1b), R₃ and R₄ each independently represent hydrogen, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position;
(B): a compound having a three- or four-membered ring cyclic ether group;
(C): a compound having a reactive group including a carbon-carbon double bond or a carbon-carbon triple bond.

8. The method for producing a cured product according to claim 7, wherein the method for producing a cured product includes a pre-curing step under a temperature condition in a range of 60° C. to 150° C. and a curing step under a temperature condition in a range of 150° C. to 240° C.

9. The method for producing a cured product according to claim 7, wherein the curable resin composition further contains a component (D) below:
(D): a curing reaction catalyst.

10. The method for producing a cured product according to claim 9, wherein the curing reaction catalyst is an acid catalyst.

11. The curable resin composition according to claim 2, further comprising a component (E) below:
(E): a filler.

12. The curable resin composition according to claim 3, further comprising a component (E) below:
(E): a filler.

13. The method for producing a cured product according to claim 8, wherein the curable resin composition further contains a component (D) below:
(D): a curing reaction catalyst.
